(19) United States
(12) Patent Application Publication
(10) Pub. No.: US 2004/0234991 A1
(43) Pub. Date: Nov. 25, 2004

(54) Method for detecting at a solid support of complexing or hybridization between at least two basic molecules based on an amplified signal at the support

(76) Inventors: Francis Garnier, Champigny-sur-Marne (FR); Bernard Mandrand, Villeurbanne (FR)

Correspondence Address: JAMES C. LYDON, 100 DAINGERFIELD ROAD, SUITE 100, ALEXANDRIA, VA, 22314, US

(21) Appl. No.: 10/485,328

(22) PCT Filed: Aug. 1, 2002
(86) PCT No.: PCT/FR02/02781

(30) Foreign Application Priority Data
Aug. 1, 2001 (FR) 0110302

(51) Int. Cl. C12Q 1/68; G01N 33/553

(52) U.S. Cl. 435/6; 436/525

(57) ABSTRACT

A method for detecting at a solid support (3) complexing or hybridization between at least two basic molecules (8 and 9), one of the molecules being initially fixed on support (3), called identifying molecule (8), and the other being in solution in liquid sample (2), called target molecule (9). A solid support for detecting complexing or hybridization between at least two different molecules, a biochip incorporating the support and use of the biochip in a diagnostic test are also disclosed. The method includes: using chemical or biological element (10), specifically bound to the formed complex or hybrid; exciting the complex or the hybrid, so that chemical or biological element (10) releases light signal (11); receiving and transforming light signal (11) into chemical signal (15) at support (3), and detecting electric signal (12) derived from chemical signal (15).

The present invention relates to the detection of biological molecules through the use of an amplification method with improved efficiency.

The recognition of a target biological molecule by a specific recognition molecule of this target biological molecule is a property widely used in the diagnostic field. Many biological molecules (DNA, RNA, protein, antibody, antigen) are thus currently detectable and quantifiable by these recognition methods. Techniques similar to those used for the detection of biological molecules are also used in the agri-food field to detect the presence of micro-organisms such as bacteria.

The first step of these detection techniques consists in fixing the recognition molecule onto a support. The support which is used to fix the recognition molecules is generally a flat or porous surface made of materials, such as:

glass, an inexpensive, inert and mechanically stable material; the surface can be covered with a Teflon grid which delimits hydrophilic and hydrophobic areas,

polymers such as polypyrrole,

silicon,

metals, notably gold and platinum.

Depending on the nature of the sought target biological molecule, DNA or protein, the recognition molecule can be made up of another DNA, an RNA, an oligonucleotide (or ODN), an antigen, or an antibody, respectively.

To fix the recognition molecules, three main types of manufacture can be singled out.

Firstly, there is a technique which consists in depositing presynthesized probes. Fixing of the probes is effected by direct transfer, by means of micropipets, micro-points or by an inkjet-type device. This technique allows for the fixing of probes of relatively large size: from 60 bases (printing) to a few hundred bases (micro-deposition):

Printing is an adaptation of the method used by inkjet printers. It relies on the propulsion of minute balls of fluids (volume <1 nl) at a rate which can reach 4000 drops/second. Printing does not involve any contact between the system releasing the fluid and the surface onto which it is deposited.

Micro-deposition consists in fixing long probes of a few hundred to several hundred bases to the surface of a glass blade. These probes are generally extracted from databases and are in the form of amplified and purified products. This technique allows chips called microarrays to be obtained, which carry approximately ten thousand DNA spots on a surface of slightly less than 4 cm². However, the use of Nylon membranes, so-called “macroarrays”, which carry amplified products, generally by PCR, with a diameter of 0.5 to 1 mm and a maximum density of 25 spots/cm², should not be forgotten. This extremely flexible technique is used by many laboratories. In the present invention, the latter technique is considered as belonging to the field of biochips.

The second technique for fixing the probes onto the support is called in situ synthesis. This technique results in the development of short probes directly at the surface of the chip. It relies on the in situ oligonucleotide synthesis invented by Edwin Southern, is based on the oligonucleotide synthesiser method, and consists in moving a reaction chamber, where the oligonucleotide elongation reaction takes place, along the glass surface.

Finally, the third technique is called photolithography, which is a method at the origin of the biochips developed by Affymetrix. Photolithography is derived from microprocessor techniques. The chip surface is modified by the fixing of photolabile chemical groups which can be activated by light. Once illuminated, these groups are capable of reacting with 3′ end of an oligonucleotide. By protecting this surface using masks of defined shapes, areas of the chip where one or the other of the 4 nucleotides are to be fixed can be illuminated and therefore selectively activated. The successive use of different masks allows the protection/reaction cycles to be alternated and therefore the oligonucleotide probes to be achieved on spots of approximately a few tens of square micrometers (μm²). This resolution allows up to several tens of thousands of spots to be created on a surface of a few square centimetres (cm²). Photolithography has advantages: extremely parallel, it allows a N-mer chip to be created in only 4×N cycles.

It is to be understood that all of these techniques can be used with the present invention.

The second step of these detection techniques consists in specifically hybridising a target molecule on the recognition molecule. It should be noted that it is also possible to form the recognition molecule/target molecule hybrid prior to fixing on the support.

The third step of these detection techniques consists either in using an already labelled target molecule or in hybridising a detection molecule on the target molecule.

According to the first alternative, the target molecule is prelabelled, so as to be able to detect hybridisation or the recognition molecule and target molecule complex after hybridisation or complexing. Several labelling techniques have been described thus far in the literature. The Applicant has already filed a patent application, WO-A-99/65925 with priority of 17 Jun. 1998, on nucleic acid labelling which is linked to the fragmentation thereof. Another conventional labelling technique consists in grafting a fluorescent label onto the target molecule. After excitation of the label, a light signal is emitted, which can be detected and analysed with fluorescent microscopy or by luminometry. Within the context of DNA detection, the detection limit by fluorescence is of the order of 10⁻⁹ to 10⁻¹⁰ mole per litre (mol/l) of labelled complementary oligonucleotides, Lü, Hua. Characterization of DNA hybridization on the optical fiber surface Colloids and Surfaces A: Physiochemical and Engineering Aspects—Vol. 175, Issues 1: p. 147-152. 15 Dec. 2000. A similar technique consists in grafting a radioactive label onto the detection probe. This technique is widely used, notably for the detection of DNA or RNA. Quantization of the signal is carried out by autoradiography, by a photographic plate which is sensitive to X-rays. Chemiluminescence is another, more recent labelling technique. Labelling is chemically effected using a chemiluminescent compound. Various chemiluminescent labels have already been described in the prior art such as luminol, lucigenine, anthracene, rubene. After probe/labelled target hybridisation, the chemiluminescent component is brought to an excited state. Depending on the label, excitation takes place by modification of the pH (luminol, lucigenine) or by the presence of peroxyoxalate (anthracene, rubene). This excited component then emits a ray in the visible spectrum, allowing the detection of the probe/target complex by luminometry. Similarly, detection can also take place by electrochemiluminescence, as is notably developed in patent WO-A-98/12539. In this case, the label is electrically excited, which leads to the emission of a photon, which is detectable by luminometry. Using electrochemistry, the detection limit is greater than when using fluorescent labels and can reach approximately 10⁻¹² to 10⁻¹³ mole of DNA sought within the context of DNA detection. Finally, another technique requires a modification of the electrochemical signature of a conjugated polymer carrying the probe, during the hybridisation of the recognition molecule/target molecule complex. Thus, in patent application FR94/5064, an electrically conductive and electroactive, conjugated polymer linked to a first biological molecule (recognition molecule) is used to specifically detect or assay a second biological molecule (target molecule). The latter is therefore electrically detected, by measuring a potential difference between the conjugated polymer not linked to the target molecule and the conjugated polymer linked to the target molecule. A comparable study is presented in patent application WO-A-00/77523.

According to the second alternative, so as to be able to detect the recognition molecule and target molecule hybrid or complex after hybridisation or complexing, the target molecule is then labelled indirectly by specific hybridisation with a detection molecule. Several labelling techniques have been described so far in the literature. Additional information in relation to this second alternative can be found in the following documents:

E. Lopez-Crapez, H. Bazin, E. Andre, J. Noletti, J. Grenier and G. Mathis—A homogeneous europium cryptate-based assay for the diagnosis of mutations by time-resolved fluorescence resonance energy transfert—Nucleic acids Res. 2001 29: e70, and

Yuzhi Zhang, Brendan D. Price, Sotirios Tetradis, Subrata Chakrabarti, Gautam Maulik and Mike Makrigiorgos—Reproductible and inexpensive probe preparation for oligonucleotide arrays—Nucleic acids Res. 2001 29: e66.

All of these techniques can be applied to the biochip field. Biochip signifies a chip or a support having a plurality of recognition areas, equipped with molecules having recognition properties, at its surface. Hereafter in the text, and through misuse of language, the term biochip is used irrespective of whether the chip is for chemical or biological analysis. The present invention finally relates to hybrids or complexes which may be used on such biochips.

The concept of biochips, more precisely of DNA chips, dates from the beginning of the 1990s. Today, this concept has been extended since protein chips are beginning to be developed. It relies on a pluridisciplinary technology incorporating micro-electronics, nucleic acid chemistry, image analysis and computing. The working principle relies on a foundation of molecular biology: the hybridisation phenomenon; i.e. matching the bases of two DNA sequences by complementarity.

The biochip method relies on the use of probes (DNA sequences representing a portion of a gene or an oligonucleotide), fixed on a solid support, on which probes a sample of nucleic acids directly or indirectly labelled with fluorochromes is allowed to act. The probes are specifically positioned on the chip and each hybridisation gives information on each represented gene. This information is cumulative, and allows the presence of a gene to be detected or the expression level of this gene in the studied tissue to be quantized. After hybridisation, the chip is washed, read by a scanner and a computer analysis of the fluorescence is carried out.

Thus, Micam chips (registered trade mark) have an effective surface of less than 2 mm². After hybridisation with labelled target DNA (reaction volume of the order of a few μl), the chip is read in fluorescence (see in this respect M. Cuzin—DNA chips: a new tool for genetic analysis and diagnostics—Transfusion Clinique et Biologique, Volume 8. Issue 3, June 2001, pages 291-296). Other biochip systems used in genetic diagnosis have also been similarly described (Vo-Dinh 1998; WO-A-99/2714; WO-A-00/43552), and can more widely be applied to the detection of target proteins or bacteria (U.S. Pat. No. 5,814,516).

However, although extremely sensitive techniques (fluorescence, electrochemistry) are used as a mode of detection, these methods have detection limits which are intrinsically associated with these techniques. Thus, the detection limit by fluorescence, i.e. the number of photons that can be distinguished corresponds to approximately 10⁻⁹ to 10⁻¹⁰ mole. Using electrochemistry, it appears that the detection limit can reach approximately 10⁻¹² to 10⁻¹³ mole. Although signal processing techniques could allow these detection limits to be pushed back by a factor of 10, even 100, the expected values are evidently well within the limits desired by researchers and practitioners to notably characterise some tens or hundreds of copies in the analysis of DNA in solution.

The invention proposes a response to all of the disadvantages of the state of the art.

To this end, the present invention relates to a method for detecting at a solid support complexing or hybridisation between at least two molecules, one of the molecules, so-called recognition molecule, being initially fixed onto the support and the other, so-called target molecule, being in solution in a liquid sample, consisting in:

using a chemical or biological element, specifically linked to the formed complex or hybrid,

exciting said complex or said hybrid, so that the chemical or biological element releases a light signal,

receiving the light signal and transforming it into a chemical signal at said support, and

detecting an electric signal resulting from the chemical signal.

According to a preferred embodiment of the invention, the chemical or biological element releases at least one photon during excitation.

According to another preferred embodiment of the invention, the amplified signal is achieved by a metal precipitate, preferably by a silver halide precipitate.

According to another preferred embodiment of the invention, detection is achieved by electric detection, and/or luminometry, and/or fluorome try, and/or radiometry, and/or photodiode.

The invention also relates to a solid support for detecting complexing or hybridisation between at least two molecules, one of the molecules, so-called recognition molecule, being in contact with the support, and the other, so-called target molecule, being in solution in a liquid sample made up of a wall of a transparent material comprising:

a first face for the adhesion of the complexes or hybrids, and

a second face carrying means for chemical amplification of a light signal derived from a chemical or biological element, specifically bound to the formed complex or hybrid.

The invention also relates to a solid support for detecting complexing or hybridisation between at least two molecules, one of the molecules, so-called recognition molecule, being in contact with the support and the other, so-called target molecule, being in solution in a liquid sample, the adhesion of the complexes or hybrids and the means for chemical amplification of a light signal coming from a chemical or biological element, which is specifically bound to the formed complex or hybrid, being on the same face of the support.

According to a preferred mode of the invention described in the previous two paragraphs, the face of the support, which carries the means for chemical amplification of a light signal, also carries detection means for the amplified signal.

According to another preferred mode of the invention, the support has a small thickness of between 0.1 μm and 100 μm, preferably between 0.5 μm and 10 μm, and still more preferably of 1 μm. Preferably, the support is substantially parallelepiped-shaped.

According to a preferred mode of the invention, the second face of the support abuts a test card, the space between both elements circumscribing the amplification means for the light signal and/or the detection means for the chemical signal.

According to another preferred mode of the invention, the chemical or biological element forms all or part of one of the two recognition or target molecules or is made up of an atom or a detection molecule initially carried by one of said two recognition or target molecules. Preferably, the element is made up of an atom or a group of atoms placed onto one of the two recognition or target molecules.

According to another preferred embodiment of the invention, all of the structurally or functionally identical recognition molecules, i.e. which hybridise or complex to the same target molecules, are grouped in a recognition area; the support can contain at least two recognition areas, preferably between a hundred and a million recognition areas and still more preferably from three hundred to a thousand recognition areas. Preferably, each recognition area is associated to amplification means which belong thereto, or are shared with all or part of the other recognition areas of the support. Preferably, the amplification means cover all or part of the support face where they are implanted.

According to another preferred mode of the invention, the amplification means for the light signal are made up of a metal layer, preferably a silver layer, which gives a metal precipitate preferably of silver in the presence of at least one photon.

According to another mode of the invention, the detection means for the amplified signal(s) are made up of an electric matrix network.

According to another mode of the invention, the support and/or the recognition areas is/are made of glass, polymer or silica.

The invention also relates to a biochip incorporating a support according to the invention described in the preceding paragraph. The biochip is preferably used in a diagnostic test.

The accompanying figures are given by way of explanatory example and are in no way limitative. They will allow the invention to be more easily understood.

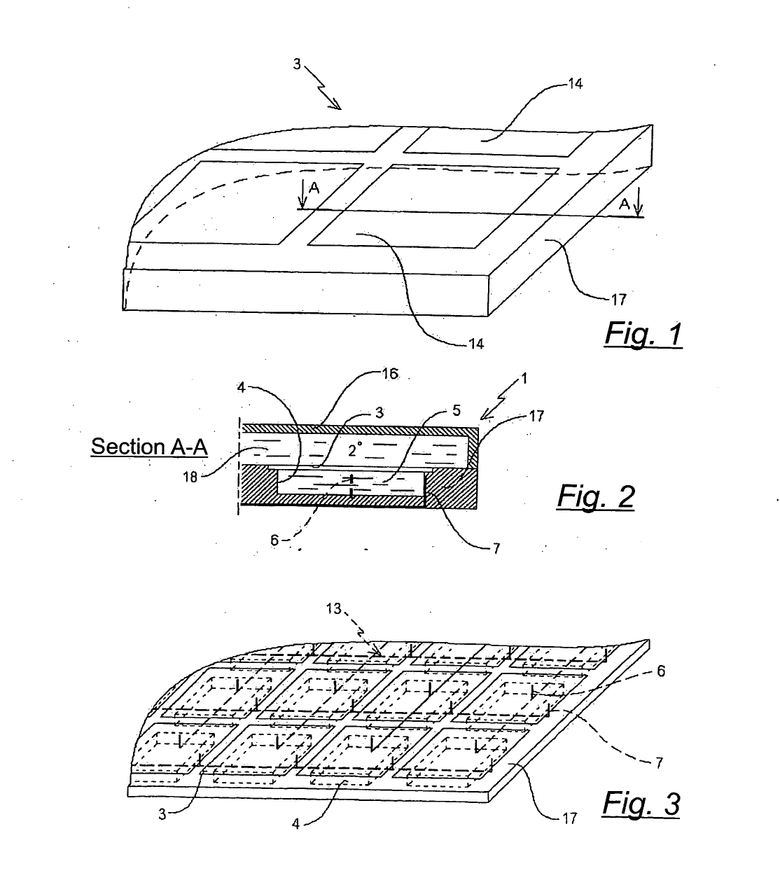

FIG. 1 shows a partial perspective view of a test card according to the invention.

FIG. 2 shows a sectional view along A-A of FIG. 1, the upper part of the test card being in place.

FIG. 3 shows a partial perspective view of a test card according to the invention, revealing the electric matrix network, seen in transparency.

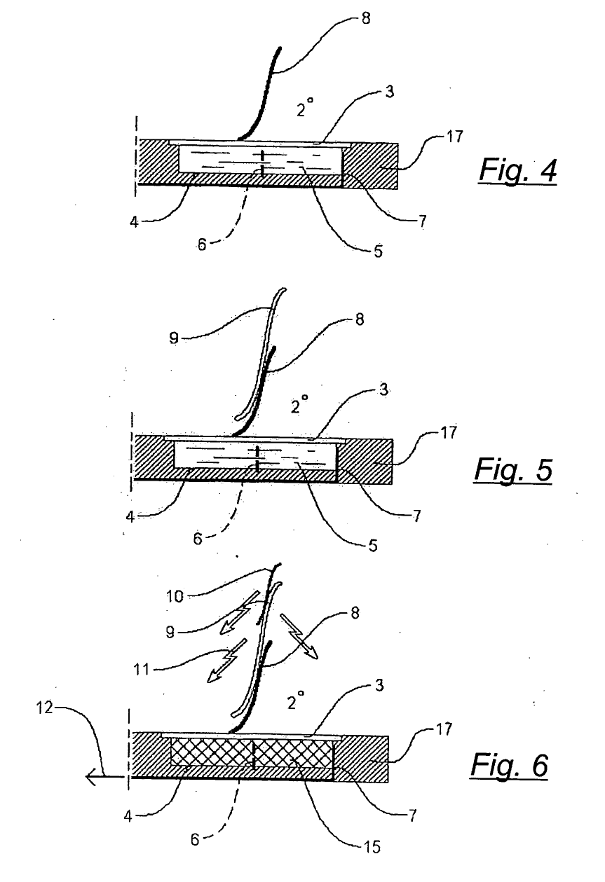

FIG. 4 shows a sectional view of a support according to the invention, after complexing of a recognition molecule on its outer surface.

FIG. 5 shows a sectional view of a support according to the invention, after hybridisation of a target molecule on the recognition molecule already complexed on the outer support surface.

Finally, FIG. 6 shows a sectional view of a support according to the invention, after hybridisation of a detection molecule on the hybrid between a target molecule and a recognition molecule already complexed on the outer support surface.

The following example is given for explanatory purposes and is in no way limitative. It will allow the invention to be more easily understood. Thus, the given example relates essentially to nucleic acids, but it is quite conceivable to use it with antibodies and antigens for example.

In general, the concept of biochips, more precisely of DNA chips, dates from the beginning of the 1990s. Today, this concept has been extended since protein chips are beginning to be developed. It relies on a pluridisciplinary technology incorporating micro-electronics, nucleic acid chemistry, image analysis and computing. The working principle relies on a foundation of molecular biology: the hybridisation phenomenon; i.e. matching the bases of two DNA sequences by complementarity.

The biochip method relies on the use of probes (DNA sequences representing a portion of a gene or an oligonucleotide), fixed on a solid support, on which probes a sample of nucleic acids directly or indirectly labelled with fluorochromes is allowed to act. The probes are specifically positioned on the chip and each hybridisation gives information on each represented gene. This information is cumulative, and allows the presence of a gene to be detected or the expression level of this gene in the studied tissue to be quantized. After hybridisation, the chip is washed, read by a scanner and a computer analysis of the fluorescence is carried out.

The support which is used to fix the probes is generally made up of a plane or porous surface made of materials, such as:

glass, an inexpensive, inert and mechanically stable material; the surface can be covered with a Teflon grid which delimits hydrophilic and hydrophobic areas,

polymers such as polypyrrole,

silicon,

metals, notably gold and platinum.

To fix the probes, hereafter also called recognition molecules, three main types of manufacture can be singled out.

Firstly, there is a technique which consists in depositing presynthesized probes. Fixing of the probes is effected by direct transfer, by means of micropipets, micro-points or by an inkjet-type device. This technique allows the fixing of probes of relatively large size: from 60 bases (printing) to a few hundred bases (micro-deposition):

Printing is an adaptation of the method used by inkjet printers. It relies on the propulsion of minute balls of fluids (volume <1 nl) at a rate which can reach 4000 drops/second. Printing does not involve any contact between the system releasing the fluid and the surface on which it is deposited.

Micro-deposition consists in fixing long probes of a few hundred to several hundred bases to the surface of a glass blade. These probes are generally extracted from databases and are in the form of amplified and purified products. This technique allows chips called microarrays to be obtained, which carry approximately ten thousand DNA spots on a surface of slightly less than 4 cm². However, the use of Nylon membranes, so-called “macroarrays”, which support amplified products, generally by PCR, with a diameter of 0.5 to 1 mm and a maximum density of 25 spots/cm², should not be forgotten. This extremely flexible technique is used by many laboratories. In the present invention, the latter technique is considered as belonging to the field of biochips.

The second technique for fixing the probes onto the support is called in situ synthesis. This technique results in the development of short probes directly at the surface of the chip. It relies upon the in situ oligonucleotide synthesis invented by Edwin Southern, is based on the oligonucleotide synthesiser method, and consists in moving a reaction chamber, where oligonucleotide elongation reaction takes place, along the glass surface.

Finally, the third technique is called photolithography, which is a method at the origin of the biochips developed by Affymetrix. Photolithography is derived from the microprocessor techniques. The chip surface is modified by the fixing of photolabile chemical groups which can be activated by light. Once illuminated, these groups are capable of reacting with the 3′ end of an oligonucleotide. By protecting this surface using masks with defined shapes, areas of the chip where one or the other of the 4 nucleotides are to be fixed can be illuminated and therefore selectively activated. The successive use of different masks allows the protection/reaction cycles to be alternated and therefore the oligo probes to be achieved on spots of approximately a few tens of square micrometers (μm²). This resolution allows up to several tens of thousands of spots to be created on a surface of a few square centimetres (cm²). Photolithography has advantages: extremely parallel, it allows a N-mer chip to be created in only 4×N cycles.

It should be understood that all of these techniques can be used with the present invention.

Methods using biochips can essentially be of two different types.

On the one hand, these can be methods for:

investigating the presence or absence of a pathogen, for example a bacterium in meat.

investigating the presence or absence of mutations, with knowledge of the gene, manufacturing oligonucleotides which represent this gene. In the presence of the biological sample, the image obtained by fluorescence allows for the discovery of whether there is a mutation and in which position it is located. In this application, the use of DNA chips is the equivalent of sequencing for a mutation diagnosis, with an enormous advantage in terms of speed.

measuring the expression level of genes in a tissue. The chip network carries a very large number of probes which correspond to the whole of the genes of the species to be studied. A sample, for example of mRNA which represents the active genes of the tissue, is hybridised. Analysis by fluorescence allows the expression level of each gene to be known.

The effectiveness of biochips has been tested on well known biological systems such as yeast (cell cycle, respiratory metabolism, fermentation, etc.). The comparison of the results obtained by the chips with those previously obtained by other approaches has shown concordance for the genes, the expression of which was already well-known in these biological systems. These works have thus allowed biochip technology to be validated.

DNA chips have opened the way to a new instrumentation in molecular biology which can even integrate different analysis steps in miniaturised form; see in this respect patent applications WO-A-00178452 with priority of 22 Jun. 1999 and FR00/10978 filed by the Applicant on 28 Aug. 2000. Furthermore, and as already indicated above, the high level of interest recently aroused by proteomics, the key discipline of post-genomics, is accompanied by the emergence of the protein chip concept. These also belong to the field of biochips according to the invention.

Recognition molecules can be, for example, oligonucleotides, polynucleotides, proteins such as antibodies or peptides, lectins or any other system of the ligand-receptor type. In particular, recognition molecules can comprise DNA or RNA fragments.

When the biochip is brought into contact with a sample to be analysed, the recognition molecules are capable of interacting, for example by hybridisation in the case where they are nucleic acids or by complexing in the case where they are an antibody and an antigen, with target molecules present in a liquid, biological sample.

Thus, by providing a biochip with a plurality of recognition areas with various, different recognition molecules, where each recognition molecule is specific to a target molecule, it is possible to detect and possibly quantize a large variety of molecules contained in the sample. It is of course evident that each recognition area comprises only one type of recognition molecules which are identical to each other.

The support-recognition molecule/target molecule-detection molecule set is a sandwich format test.

Sandwich format tests are widely used in diagnosis, whether in molecular diagnostics, for example the ELOSA test (Enzyme-Linked Oligo-Sorbent Assay), or in immunological diagnostics, for example the ELISA test (Enzyme-Linked Immuno-Sorbent Assay). In general, they include a recognition molecule, such as a nucleic acid probe or an antigen (case of an antigen sandwich) or an antibody (case of an antibody sandwich), which is used to capture a target, which will be made up of a nucleic acid probe or an antibody or an antigen respectively. This recognition molecule is fixed onto a solid support in a manner known by the person skilled in the art, either by adsorption, direct coupling, or by means of an intermediary protein, such as avidin or protein A for example. The recognition molecule and target molecule set is then detected by a detection molecule, which will be made up of a nucleic acid probe, an antibody or an antigen, respectively. This detection molecule carries or can subsequently be connected to a label, which label is necessary to allow detection and/or quantization. The detection molecule, whether it is connected or not yet connected with a label will always be called a detection molecule.

Hereafter, the fixing of a nucleic acid onto another nucleic acid is called hybridisation, whereas the fixing of an antibody onto an antigen is called complexing.

The tests which are currently available are tests such as those developed by one of the Applicants for immunoassays or the DNA chips perfected by the Affymetrix company (“Accessing Genetic Information with High-Density DNA arrays”, M. Shee et al., Science, 274, 610-614. “Light-generated oligonucleotide arrays for rapide DNA sequence analysis”, A. Caviani Pease et al., Proc. Natl. Acad. Sci. USA, 1994, 91, 5022-5026), for molecular diagnostics. In this technology, the capture probes are generally of a reduced size, around twenty nucleotides.

In the ELOSA field, i.e. the detection of nucleic acids, see in this respect patent application WO-A-91/19812 filed by the Applicant, a capture oligonucleotide, a nucleic acid target, which is either DNA or RNA, and a detection oliginucleotide are defined in the same way. The capture and detection oligonucleotides are complementary of a part of the target but at regions of the target which are structurally and physically different respectively, such that the capture and detection oligonucleotides cannot hybridise with each other.

Whether in molecular diagnostics or immunoassays, the detection elements carry a label which allows for the detection and/or the quantization of the target. In the state of the art, various labels have been developed with the permanent aim of improving sensitivity. They can either be radioactive, enzymatic, fluorescent or in the form of microparticles or nanoparticles.

Labelling means the fixing of a label capable of directly or indirectly generating a detectable signal. A non-limiting list of these labels follows:

those enzymes which produce a detectable signal for example by colorimetry, fluorescence, luminescence, such as horseradish peroxydase, alkaline phosphatase, β-galactosidase, glucose-6-phosphate dehydrogenase,

those chromophores such as fluorescent, luminiscent, colorant components,

those groups with an electronic density detectable by electronic microscopy or by their electric property such as conductivity, amperometry, voltametry, impedance,

those detectable groups, the molecules of which, for example, are of a sufficient size to induce detectable modifications in their physical and/or chemical characteristics, such detection can be achieved by optical methods such as diffraction, surface plasmon resonance, surface variation, contact angle variation or physical methods such as atomic force spectroscopy, tunnel effect,

those radioactive molecules such as ³²P, ³⁵S or ¹²⁵I.

In the present invention, the label is essentially made up of a group able to provide a photon.

Indirect systems can also be used, for example, ligands capable of reacting with an anti-ligand. The ligand/anti-ligand couples are well known by the person skilled in the art, which is the case for example with the following couples:

biotin/streptavidin,

hapten/antibody,

antigen/antibody,

peptide/antibody,

sugar/lectin,

polynucleotide/polynucleotide complementary.

In this case, it is the ligand which carries the linking agent. The anti-ligand can be directly detectable by the labels described in the preceding paragraph or can itself be detectable by a ligand/anti-ligand.

These indirect detection systems can lead, under certain conditions, to an amplification of the signal. This signal amplification technique is well known by the person skilled in the art, and reference can be made to earlier patent applications FR98/10084 or WO-A-95/08000 by the Applicant or to article J. Histochem. Cytochem. 45: 481-491, 1997.

All of these above-mentioned elements are capable of being incorporated into the invention so as to further improve the performances thereof.

If reference is now made to the figures, it is noted in FIG. 1 that the invention relates to a test card 1, of which only the first so-called base part 17 is shown. This 1 comprises on one of its flat faces a number of recognition areas 14, which together make up a support 3. Support 3 greatly absorbs the ultraviolet but is transparent in the visible spectrum. In order to do so, said glass support 3 is precharged with lead.

As shown in FIG. 2, support 3 is itself deposited within test card 1, which can also be made of glass, or of an appropriate plastic. This card 1, such as shown in FIG. 2, is made up of two parts, the first so-called base part 17, already mentioned above, and a second part made up of a cover 16, which delimit, in conjunction with supports 3, a space 18 where a liquid sample to be tested 2 can be introduced via any means from the prior art known by the person skilled in the art.

This first so-called base part 17 and each support 3 together delimit a tank 4 where a signal amplification means 5 is present. The nature and operation of this signal amplification means 5 will be described later.

On either side of tank 4, the presence of an upstream electric terminal 6 and a downstream electric terminal 7, which have no direct contact with each other, is noted. By contrast, said signal amplification means 5 is in contact with both upstream electric terminal 6 and downstream electric terminal 7.

Base part 17 will be better understood from the inside in FIG. 3. In this figure, it comprises a number of supports 3, each connected to a tank 4 and a signal amplification means 5, itself connected to an upstream electric terminal 6 and a downstream electric terminal 7. The set of upstream electric terminals 6 is perpendicularly positioned to the set of downstream electric terminals 7 without any contact between these two sets, and together they form an electric matrix network 13.

In these FIGS. 4 to 6, and for the sake of facilitating the understanding of the invention, cover 16 has not been shown and recognition molecule 8, as well as the other molecules mentioned hereafter which cooperate therewith, have only been shown as a single example and in a stylised and enlarged manner.

In FIG. 4, a recognition molecule 8 is deposited onto one of recognition areas 14 of a glass support 3, according to a conventional micro-deposit technique, already mentioned above. It should be understood that this fixing is carried out prior to the introduction of sample 2 to be tested into space 18.

FIG. 5 shows the following step which consists in allowing hybridisation of a target molecule 9 on a recognition molecule 8. This hybridisation causes no modification at base part 17 of card 1, including support 3.

Finally, FIG. 6 shows the last biological step. It consists in allowing hybridisation of a third so-called detection molecule 10 on the hybrid made up by recognition molecule 8 and target molecule 9. It should be understood that it is also possible that this detection molecule 10 is an integral part of target molecule 9, in which case this step is unnecessary since detection can be achieved on formation of hybrid 8 and 9.

Detection molecule 10 is a molecular group capable of specifically binding onto target molecule 9 or onto hybrid 8 and 9, and capable of producing at least one photon in order to produce a light signal 11.

This hybridisation does not cause any modification at support 3. By contrast, it causes a modification at tank 4 of base part 17 of card 1, and more precisely at chemical amplification means 5 for light signal 11, which is made up of a silver halide (AgX, such as AgCl, AgBr, etc.). Thus, light signal 11 will bring about a silver precipitation 15 within said tank 4. This precipitation will therefore close the electric circuit between upstream and downstream electric terminals 6 and 7, which will allow an electric signal 12 to be conducted.

Thus, electric network 13 of FIG. 3 will allow, via the matrix grid of micro-circuit lines 6 and columns 7, a precipitate, corresponding in its coordinates to recognition molecules 8 having specifically hybridised target molecules 9, to appear or not at each intersection.

It should be noted that target molecule 9 is labelled with a fluorescent chemical element 10, according to a conventional labelling technique known by the person skilled in the art.

Formed hybrid 8 and 9, bound to fluorescent chemical element 10, is subjected to total excitation by ultraviolet radiation. Local emissions take place in the visible spectrum from hybrid 8 and 9, forming a light signal 11. Unlike the excitation ultraviolet radiations, this light signal 11 passes through support 3, which is transparent in the visible and is transformed into a chemical signal 15 by the precipitation of silver halide 5. The interaction of this light signal 11 and of silver halide 5 leads to the formation in tank 4 of chemical signal 15 including silver microcrystallites with respect to formed hybrid 8 and 9. These silver microcrystallites establish a contact between the line and column of the matrix grid. Thus, the conductivity established between the line and column, linked to the size of the microcrystallites formed is directly linked to the quantity of light emitted, and therefore to the quantity of hybridised recognition molecules 8 and target molecules 9.

### REFERENCES

1. Test card

2. Liquid sample to be tested

3. Solid support or recognition area

4. Tank

5. Amplification means for a signal or metal solution or silver solution

6. Upstream electric terminal

7. Downstream electric terminal

8. Recognition molecule

9. Target molecule

10. Chemical or biological element or detection molecule

11. Light signal

12. Electric signal

13. Electric network

14. Recognition areas of support 3

15. Chemical signal or metal precipitate preferably of silver

16. Cover of card 1

17. Base part of card 1

18. Space circumscribed by a support 3, cover 16 and base part 17

1-20. (canceled).

21. A method for detecting a solid support complexing or hybridisation between at least two molecules, one of the molecules, so-called recognition molecule, being initially fixed onto the support, and the other, so-called target molecule, being in solution in a liquid sample, comprising:
using a chemical or biological element, specifically bound to the formed complex or hybrid,
exciting said complex or said hybrid, so that the chemical or biological element releases a light signal,
receiving the light signal and transforming it into a chemical signal at said support, and
detecting an electrical signal, resulting from the chemical signal.

22. The method of claim 21, wherein, during excitation, the chemical or biological element releases at least one photon.

23. The method of claim 21, wherein the chemical signal is achieved by a metal precipitate.

24. The method of claim 21, wherein detection is achieved by at least one method selected from the group consisting of electric detection, luminometry, fluorometry, radiometry, and photodiode.

25. A solid support for detecting complexing or hybridisation between at least two molecules, one of the molecules, so-called recognition molecule, being in contact with the support and the other, so-called target molecule, being in solution in a liquid sample, wherein it is made up of a wall of a transparent material comprising:
a first face for the adhesion of the complexes or hybrids, and
a second face delimiting chemical amplification means for a light signal derived from a chemical or biological element, specifically bound to the formed complex or hybrid.

26. A solid support for detecting complexing or hybridisation between at least two molecules, one of the molecules, so-called recognition molecule, being in contact with the support and the other, so-called target molecule, being in solution in a liquid sample, wherein the adhesion of the complexes or hybrids and the chemical amplification means for a light signal derived from a chemical or biological element, which is specifically bound to the formed complex or hybrid, are on the same face of said support.

27. The support of claim 25, wherein the face of the support, which carries the chemical amplification means for a light signal, also carries detection means for the chemical signal.

28. The support of claim 25, wherein the support has a thickness of between 0.1 μm and 100 μm.

29. The support of claim 28, wherein the support is substantially parallelepiped-shaped.

30. The support of claim 25, wherein the second face of the support abuts a test card, the space between both elements circumscribing the amplification means for a light signal and/or the detection means for the chemical signal.

31. The support of claim 25, wherein the chemical or biological element forms all or part of one of the two recognition or target molecules or is made up of an atom or a detection molecule initially carried by one of said two recognition or target molecules.

32. The support of claim 25, wherein the chemical or biological element is made up of an atom or a group of atoms placed onto one of the two recognition or target molecules.

33. The support of claim 25, wherein all of the structurally or functionally identical recognition molecules are grouped in a recognition area; the support can contain at least two recognition areas.

34. The support of claim 33, wherein each recognition area is associated to amplification means which:
belong thereto, or
are shared with all or part of the other recognition areas of the support.

35. The support of claim 33, wherein the amplification means cover all or part of the face of the support where they are implanted.

36. The support of claim 25, wherein the amplification means for the light signal are made up of a metal layer, which gives a metal precipitate, in the presence of at least one photon.

37. The support of claim 27, wherein the detection means for the chemical signals are made up of an electric matrix network.

38. The support of claim 25, wherein the support and/or the recognition areas is/are made of glass, polymer or silica.

39. A biochip incorporating a support according to claim 25.

40. (Canceled).

41. The support of claim 26, wherein the face of the support, which carries the chemical amplification means for a light signal, also carries detection means for the chemical signal.

42. The support of claim 26, wherein the support has a thickness of between 0.1 μm and 100 μm.

43. The support of claim 42, wherein the support is substantially parallelepiped-shaped.

44. The support of claim 26, wherein a second face of the support abuts a test card, the space between both elements circumscribing the amplification means for a light signal and/or the detection means for the chemical signal.

45. The support of claim 26, wherein the chemical or biological element forms all or part of one of the two recognition or target molecules or is made up of an atom or a detection molecule initially carried by one of said two recognition or target molecules.

46. The support of claim 26, wherein the chemical or biological element is made up of an atom or a group of atoms placed onto one of the two recognition or target molecules.

47. The support of claim 26, wherein all of the structurally or functionally identical recognition molecules, are grouped in a recognition area; the support can contain at least two recognition areas.

48. The support of claim 47, wherein each recognition area is associated to amplification means which:
belong thereto, or
are shared with all or part of the other recognition areas of the support.

49. The support of claim 47, wherein the amplification means cover all or part of the face of the support where they are implanted.

50. The support of claim 26, wherein the amplification means for the light signal are made up of a metal layer, which gives a metal precipitate, in the presence of at least one photon.

51. The support of claim 41, wherein the detection means for the chemical signals are made up of an electric matrix network.

52. The support of claim 26, wherein the support and/or the recognition areas is/are made of glass, polymer or silica.

53. A biochip incorporating a support according to claim 26.

54. (Canceled).

55. The method of claim 23, wherein the chemical signal is achieved by a silver halide precipitate.

56. The support of claim 28, wherein the support has a thickness of between 0.5 μm and 10 μm.

57. The support of claim 56, wherein the support has a thickness of 1 μm.

58. The support of claim 25, wherein said support contains between a hundred and a million recognition areas.

59. The support of claim 58, wherein said support contains between three hundred and a thousand recognition areas.

60. The support of claim 36, wherein the amplification means for the light signal are made up of a silver layer, which gives a silver precipitate, in the presence of at least one photon.

61. The support of claim 42, where the support has a thickness of between 0.5 μm and 10 μm.

62. The support of claim 61, where the support has a thickness of 1 μm.

63. The support of claim 47, wherein said support contains between a hundred and a million recognition areas.

64. The support of claim 63, wherein said support contains from three hundred to a thousand recognition areas.

65. The support of claim 50, wherein the amplification means for the light signal are made up of a silver layer, which gives a silver precipitate, in the presence of at least one photon.
